(19) United States
(12) Patent Application Publication
(10) Pub. No.: US 2026/0275369 A1
(43) Pub. Date: Sep. 17, 2026

(54) PLANTS WITH BENEFICIAL HERITABLE TRAITS

(71) Applicant: Aferna, Inc., Saint Louis, MO (US)

(72) Inventors: Jon Schmuke, Saint Louis, MO (US); Ye Fu, Natick, MA (US)

(21) Appl. No.: 19/472,284

(22) PCT Filed: Apr. 3, 2024
(86) PCT No.: PCT/US2024/022880

§ 371 Date: Oct. 3, 2025

Related U.S. Application Data
(60) Provisional application No. 63/494,446, filed on Apr. 5, 2023.

(51) Int. Cl. C12N 15/82; A01H 1/00; C12N 9/02

(52) U.S. Cl. CPC C12N 15/8261; A01H 1/12; C12N 9/0071

(57) ABSTRACT

The present invention is directed towards plants and methods of producing plants exhibiting one or more useful traits such as increased yield, increased biomass, drought tolerance, salinity tolerance, increased lignin production, biotic stress resistance, abiotic stress resistance, enhanced growth rate, or beneficial root architecture. In particular, the present invention is directed to plants, plant cells, leaves, stems, flowers, and seeds without genetic modification exhibiting useful traits that can confer one or more useful traits to successive generations including cells, leaves, stems, flowers, and seeds, methods of producing and using the plants and plant parts, and products of those plant and plant parts, including processed products such as foodstuffs, feed or meal.

### CROSS-REFERENCE TO RELATED APPLICATIONS

This application claims priority to U.S. Provisional Application No. 63/494446, filed Apr. 5, 2023, the entire contents of which is incorporated by reference herein.

### FIELD OF INVENTION

A permissive chromatin state allows for an increase in the transcription of proteins resulting in various advantageous plant phenotypes such as increased yield, growth rate, biomass, vigor, oil content, seed yield, fiber yield, fiber quality, fiber length, photosynthetic capacity, and/or abiotic stress tolerance. The disclosure relates to plants having a permissive chromatin state that is persistent and heritable to a set of subsequent generations, the subsequent generations having no genetic modification.

### BACKGROUND

It has been documented that a permissive state of chromatin allows for increased transcription of proteins. This increase in transcription in plants results in a multitude of beneficial phenotypes including increases in yield, growth rate, biomass, vigor, oil content, seed yield, fiber yield, fiber quality, fiber length, photosynthetic capacity, and/or abiotic stress tolerance.

### SUMMARY OF THE INVENTION

Plants and methods for producing plants exhibiting useful traits, methods for identifying plants without genetic modification exhibiting useful traits and methods for obtaining plants without genetic modification exhibiting useful traits that can confer a useful traits to successive generations including cells, leaves, stems, flowers, and seeds, methods of using the plants and plant parts, and products of those plant and plant parts, including processed products such as foodstuffs, feed or meal are provided herein. The surprising discovery has been made that methylation may be achieved without genetic insertion, and through various forms of editing, and such change persists through a set of generations of plants. Thus, plant progeny having no genetic editing nevertheless express beneficial phenotypes for crop production.

In certain embodiments, described herein are methods to generate plants exhibiting a useful trait comprising the steps of: (a) expressing a(n) RNA demethylase gene(s) in a first parental plant cell: (b) outcrossing the parental plant of step (a), progeny of said parental plant of step (a), a plant obtained from said plant cell of step (a), or progeny of a plant obtained from said plant cell of step (a) to a second plant wherein the RNA demethylase gene is not expressed; (c) screening a population of progeny plants obtained from said outcross of step (b) from at least one useful trait, wherein a portion of said population of plants express the RNA demethylase gene; and (d) selecting a progeny plant comprising said trait that does not express the RNA demethylase gene, wherein said trait is heritable and reversible, are provided.

In other embodiments, methods for producing a plant exhibiting a useful trait comprising: (a) expressing a(n) RNA demethylase gene(s) in a first parental plant cell; (b) outcrossing the parental plant of step (a), progeny of said parental plant of step (a), a plant obtained from said plant cell of step (a), or progeny of a plant obtained from said plant cell of step (a) to a second plant wherein the RNA demethylase gene is not expressed: (c) screening a population of progeny plants obtained from said outcross of step (b) from at least one useful trait, wherein a portion of said population of plants express the RNA demethylase gene; and (d) selecting a progeny plant comprising said trait that does not express the RNA demethylase gene, wherein said trait is heritable and reversible.

In some embodiments, the plants and the methods of making those plants have a useful trait wherein the method further comprises the step of producing seed from: i) a selected progeny plant of step (d); ii) an out-crossed progeny plant of step (d), or, iii) from both of a selected and out-crossed progeny plant of step (d).

In some embodiments the RNA demethylase gene is an FTO.

In certain embodiments the plant exhibits a useful trait that is increased yield, increased biomass, drought tolerance, salinity tolerance, increased lignin production, biotic stress resistance, abiotic stress resistance, or enhanced growth rate.

In other embodiments, the plant expresses the RNA demethylase through the insertion of an RNA demethylase gene via Agrobacterium, gene gun, callous transformation, CRISPR, TALENS, or zinc fingers. Other methodologies for inserting the RNA demethylase gene are known by those skilled in the art.

In some embodiments plant expresses the RNA demethylase through the use of base-pair editing. Base-pair editing is achieved through, DNA, RNA or RNA base editing, as provided. In yet other embodiments the plant is made using RNA or RNP base editing which further comprises enzymes that target the RNA demethylase and are expressed transiently.

In certain embodiments, an artificial nucleic acid construct, wherein the artificial nucleic acid construct comprises an RNA demethylase, the artificial nucleic acid independently comprising between 400 and 1100 nucleotides or nucleosides or a combination thereof; and when integrated within the plant, the artificial nucleic acid construct is configured to facilitate an epigenetic modification of at least one base of a ribonucleotide or ribonucleoside in a ribonucleic acid of the plant, as provided. Further, the artificial nucleic acid construct may be an FTO gene, as in some embodiments. In some embodiments, the progeny of the plant does not comprise the artificial nucleic acid construct, but comprising the epigenetic modification of at least one base of a ribonucleotide or ribonucleoside in a ribonucleic acid of said plant, as provided.

In preferred embodiments, the plant or method of making a plant wherein the plant is a crop. In embodiments the crop is soy, maize, wheat, rice, camelina, canola, sugarcane, cucurbits, strawberries, orsetaria.

Also provided herein are methods for producing seed that comprise harvesting seed from any of the aforementioned plants or crop plants of the invention. In certain embodiments, methods for producing a lot of seed comprising the steps of selecting a population of plants or crop plants of the invention, growing the selected plants, and harvesting seed therefrom are provided. In certain embodiments, the harvested seed or a plant obtained therefrom exhibits the improvement in at least one useful trait.

Also provided herewith are methods of using any of the aforementioned plants or crop plants of the invention that comprise any of the improved traits, where the methods comprise growing, propagating, or cultivating the plants or crop plants of the invention that exhibit the improved trait. Methods of obtaining improved yields that comprise harvesting any plant part including a seed of any of the plants or crop plants of the invention are also provided. In certain embodiments, the harvested seed or a plant obtained therefrom exhibits the improvement in at least one useful trait.

Also provided herein are plants, plant parts, including but not limited to, seeds, leaves, stems roots, and flowers, or products of the plants, or plant parts including but not limited to seeds, that comprise a chromosomal modification associated with a useful trait or a nucleotide or ribonucleotide alteration associated with a useful trait. In certain embodiments, the plant part can comprise a non-regenerable plant part or non-regenerable portion of a plant part. In certain embodiments, the products can be processed products that include, but are not limited to, a feed or a meal obtained from a plant part. In certain embodiments, the plants seed, or products thereof that exhibit useful traits caused by a chromosomal modification exhibits an improvement in at least one useful trait in comparison to a plant, plant parts including seeds, or products of the plants or seeds, that do not comprise the nucleotide or ribonucleotide modification.

Also provided herein is a method for producing a plant exhibiting one or more of increased yield, increased biomass, drought tolerance, salinity tolerance, increased lignin production, biotic stress resistance, abiotic stress resistance, enhanced growth rate, or beneficial root architecture, the method comprising: (a) expressing a(n) RNA demethylase gene(s) in a first parental plant cell: (b) outcrossing the parental plant of step (a) with a progeny of said parental plant of step (a) or a plant obtained from said plant cell of step (a), or progeny of a plant obtained from said plant cell of step (a) to a second plant wherein the RNA demethylase gene is not expressed; (c) screening a population of progeny plants obtained from said outcross of step (b) for one or more of increased yield, increased biomass, drought tolerance, salinity tolerance, increased lignin production, biotic stress resistance, abiotic stress resistance, enhanced growth rate, or beneficial root architecture, wherein a portion of said population of plants expresses the RNA demethylase gene; and (d) selecting a progeny plant comprising one or more of increased yield, increased biomass, drought tolerance, salinity tolerance, increased lignin production, biotic stress resistance, abiotic stress resistance, enhanced growth rate, or beneficial root architecture that does not express the RNA demethylase gene, wherein one or more of increased yield, increased biomass, drought tolerance, salinity tolerance, increased lignin production, biotic stress resistance, abiotic stress resistance, enhanced growth rate, or beneficial root architecture is heritable and reversible. In some embodiments, the method further comprises the step of producing seed from: i) a selected progeny plant of step (d); ii) an out-crossed progeny plant of step (d), or, iii) from both of selected and out-crossed progeny plant of step (d). In some embodiments, the RNA demethylase gene is an FTO.

Also provided herein are plants, plant parts, including but not limited to, seeds, leaves, stems roots, and flowers, or products of the plants, or plant parts including but not limited to seeds, that express the RNA demethylase through the insertion of an RNA demethylase gene via agrobacterium, gene gun, callous transformation, CRISPR, TALENs, or zinc fingers. In some embodiments, the plants, plant parts, including but not limited to, seeds, leaves, stems roots, and flowers, or products of the plants, or plant parts including but not limited to seeds, express the RNA demethylase through the use of base-pair editing. In some embodiments, base-pair editing is achieved through, DNA, RNA or RNA base editing. In some embodiments, the RNA or RNA base editing further comprises enzymes that target the RNA demethylase and are expressed transiently.

Also provided herein is an artificial nucleic acid construct, wherein the artificial nucleic acid construct comprises an RNA demethylase, the artificial nucleic acid independently comprising between 400 and 1100 nucleotides or nucleosides or a combination thereof; and when integrated within a plant, the artificial nucleic acid construct is configured to facilitate an epigenetic modification of at least one base of a ribonucleotide or ribonucleoside in a ribonucleic acid of the plant. In some embodiments, the RNA demethylase is an FTO gene. In some embodiments, the progeny of said plant does not comprise the artificial nucleic acid construct but comprises the epigenetic modification of at least one base of a ribonucleotide or ribonucleoside in a ribonucleic acid of said plant. In some embodiments, the plant is a crop. In some embodiments, the crop is soy, maize, wheat, rice, camelina, canola, sugarcane, cucurbits, strawberries, or setaria. In some embodiments, the artificial nucleic acid construct is introduced into the plant by a callous phase transformation.

### DETAILED DESCRIPTION

This description is not intended to be a detailed catalog of all the different ways in which the invention may be implemented, or all the features that may be added to the invention. For example, features illustrated with respect to one embodiment may be incorporated into other embodiments. and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Nucleotide sequences provided herein are presented in the 5′ to 3′ direction, from left to right and are presented using the standard code for representing nucleotide bases as set forth in 37 C.F.R. § § 1.821-1.825 and the World Intellectual Property Organization (WIPO) Standard ST. 25, for example: adenine (A), cytosine (C), thymine (T), and guanine (G).

Amino acids are likewise indicated using the WIPO Standard ST.25, for example: alanine (Ala; A), arginine (Arg; R), asparagine (Asn; N), aspartic acid (Asp; D), cysteine (Cys; C), glutamine (Gin; Q), glutamic acid (Glu; E), glycine (Gly; G), histidine (His; H), isoleucine (lie; 1), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V). As used herein, an “X” or “Xaa” in an amino acid sequence denotes that the amino acid in that position can be any of the 20 known amino acid or can be any of the enumerated amino acids recited herein.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

### Definitions

As utilized in accordance with the present disclosure, the following terms, unless otherwise indicated, shall be understood to have the following meanings:

As used herein and in the appended claims, the singular forms “a,” “an,” and “the” include plural reference unless the context clearly dictates otherwise. Thus, for example, reference to “a plant” is a reference to one or more plants and includes equivalents thereof known to those skilled in the art, and so forth.

As used herein, the word “and/or” refers to and encompasses any and all possible combinations of one or more of the associated listed items, as well as the lack of combinations when interpreted in the alternative, “or.”

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). With regard to a temperature the term “about”means ±1 °C, preferably ±0.5 °C. Where the term “about” is used in the context of this invention (e.g., in combinations with temperature or molecular weight values) the exact value (i.e., without “about”) is preferred.

As used herein, phrases such as “between about X and Y”, “between about X and about Y”, “from X to Y” and “from about X to about Y” (and similar phrases) should be interpreted to include X and Y, unless the context indicates otherwise.

The terms “comprising,” “including,” and “having” are intended to be inclusive and mean that there may be additional elements other than the listed elements.

A “gene” is a defined region that is located within a genome and comprises a coding nucleic acid sequence and typically also comprises other, primarily regulatory, nucleic acids responsible for the control of the expression, that is to say the transcription and translation, of the coding portion. A gene may also comprise other 5′ and 3′ untranslated sequences and termination sequences. Further elements that may be present are, for example, introns. The regulatory nucleic acid sequence of the gene may not normally be operatively linked to the associated nucleic acid sequence as found in nature and thus would be a chimeric gene.

A “nucleic acid molecule” or “nucleic acid sequence” is a segment of single-or double-stranded DNA or RNA that can be isolated from any source. In the context of the invention, the nucleic acid molecule is typically a segment of DNA. In some embodiments, the nucleic acid molecules of the invention are isolated nucleic acid molecules.

Operably linked. Combining two or more molecules in such a fashion that in combination they function properly in a plant cell. For instance, a promoter is operably linked to a structural gene when the promoter controls transcription of the structural gene

Plant. As used herein, the term “plant” includes but is not limited to angiosperms and gymnosperms such as potato, tomato, tobacco, alfalfa, lettuce, carrot, strawberry, sugarbeet, cassava, sweet potato, soybean, maize, turf grass, wheat, rice, barley, sorghum, oat, oak, eucalyptus, walnut, and palm. Thus, a plant may be a monocot or a dicot. The word “plant,” as used herein, also encompasses plant cells, seed, plant progeny, propagule whether generated sexually or asexually, and descendants of any of these, such as cuttings or seed. Plant cells include suspension cultures, callus, embryos, meristematic regions, callus tissue, leaves, roots, shoots, gametophytes, sporophytes, pollen, seeds and microspores. Plants may be at various stages of maturity and may be grown in liquid or solid culture, or in soil or suitable media in pots, greenhouses or fields. Expression of an introduced leader, trailer or gene sequences in plants may be transient or permanent. A “selected plant species” may be, but is not limited to, a species of any one of these “plants.”

As used herein the phrase “plant yield” refers to the amount (e.g., as determined by weight or size) or quantity (numbers) of tissues or organs produced per plant or per growing season. Hence increased yield could affect the economic benefit one can obtain from the plant in a certain growing area and/or growing time.

It should be noted that a plant yield can be affected by various parameters including, but not limited to, plant biomass; plant vigor, growth rate; seed yield; seed or grain quantity; seed or grain quality; oil yield: content of oil, starch and/or protein in harvested organs (e.g., seeds or vegetative parts of the plant); number of flowers (florets) per panicle (expressed as a ratio of number of filled seeds over number of primary panicles); harvest index; number of plants grown per area; number and size of harvested organs per plant and per area; number of plants per growing area (density); number of harvested organs in field; total leaf area; carbon assimilation and carbon partitioning (the distribution/allocation of carbon within the plant); resistance to shade; number of harvestable organs (e.g. seeds), seeds per pod, weight per seed; and modified architecture [such as increase stalk diameter, thickness or improvement of physical properties (e.g. elasticity)].

Plant Parts. As used herein, the term “plant parts” (or a potato plant, or a part thereof) includes but is not limited to protoplast, leaf, stem, root, root tip, anther, pistil, seed, embryo, pollen, ovule, cotyledon, hypocotyl, flower, tuber, eye, tissue, petiole, cell, meristematic cell, and the like.

A “plant cell” as used herein refers to any plant cell and can comprise a cell at the plant surface or internal to the plant plasma membrane, for example, an epidermal cell, a trichome cell, a xylem cell, a phloem cell, a sieve tube element, or a companion cell.

A “polynucleotide” refers to a polymer composed of many nucleotide monomers covalently bonded in a chain. Such “polynucleotides” includes DNA, RNA, modified oligo nucleotides (e.g., oligonucleotides comprising bases that are not typical to biological RNA or DNA, such as 2′-0-methylated oligonucleotides), and the like. In some embodiments, a nucleic acid or polynucleotide can be single-stranded, double-stranded, multi-stranded, or combinations thereof. Unless otherwise indicated, a particular nucleic acid or polynucleotide of the present invention optionally comprises or encodes complementary polynucleotides, in addition to any polynucleotide explicitly indicated.

As used herein, the term “recombinant” refers to a form of nucleic acid (e.g., DNA or RNA) or protein or an organism that would not normally be found in nature and as such was created by human intervention. As used herein, a “recombinant nucleic acid molecule” is a nucleic acid molecule comprising a combination of polynucleotides that would not naturally occur together and is the result of human intervention, e.g., a nucleic acid molecule that is comprised of a combination of at least two polynucleotides heterologous to each other, or a nucleic acid molecule that is artificially synthesized, for example, a polynucleotide synthesize using an assembled nucleotide sequence, and comprises a polynucleotide that deviates from the polynucleotide that would normally exist in nature, or a nucleic acid molecule that comprises a transgene artificially incorporated into a host cell's genomic DNA and the associated flanking DNA of the host cell's genome. Another example of a recombinant nucleic acid molecule is a DNA molecule resulting from the insertion of a transgene into a plants genomic DNA, which may ultimately result in the expression of a recombinant RNA or protein molecule in that organism. As used herein, a “recombinant plant” is a plant that would not normally exist in nature, is the result of human intervention, and contains a transgene or heterologous nucleic acid molecule incorporated into its genome. As a result of such genomic alteration, the recombinant plant is distinctly different from the related wild-type plant.

Regulatory sequences. Refers to those sequences which are standard and known to those in the art that may be included in the expression vectors to increase and/or maximize transcription of a gene of interest or translation of the resulting RNA in a plant system. These include, but are not limited to, promoters, peptide export signal sequences, introns, polyadenylation, and transcription termination sites. Methods of modifying nucleic acid constructs to increase expression levels in plants are also generally known in the art (see, e.g. Rogers et al., 260 J. Biol. Chem. 3731-38, 1985; Cornejo et al., 23 Plant Mol. Biol. 567:81, 1993). In engineering a plant system to affect the rate of transcription of a protein, various factors known in the art, including regulatory sequences such as positively or negatively acting sequences, enhancers and silencers, as well as chromatin structure may have an impact. The present invention provides that at least one of these factors may be utilized in engineering plants to express a protein of interest. The regulatory sequences of the present invention are native genetic elements, i.e., are isolated from the selected plant species to be modified.

As used herein the phrase “seed yield” refers to the number or weight of the seeds per plant, seeds per pod, or per growing area or to the weight of a single seed, or to the oil extracted per seed. Hence seed yield can be affected by seed dimensions (e.g., length, width, perimeter, area and/or volume), number of (filled) seeds and seed filling rate and by seed oil content. Hence increase seed yield per plant could affect the economic benefit one can obtain from the plant in a certain growing arca and/or growing time; and increase seed yield per growing area could be achieved by increasing seed yield per plant, and/or by increasing number of plants grown on the same given area.

The term “seed” (also referred to as “grain” or “kernel”) as used herein refers to a small embryonic plant enclosed in a covering called the seed coat (usually with some stored food), the product of the ripened ovule of gymnosperm and angiosperm plants which occurs after fertilization and some growth within the mother plant.

The phrase “oil content” as used herein refers to the amount of lipids in a given plant organ, either the seeds (seed oil content) or the vegetative portion of the plant (vegetative oil content) and is typically expressed as percentage of dry weight (10 % humidity of seeds) or wet weight (for vegetative portion).

It should be noted that oil content is affected by intrinsic oil production of a tissue (e.g., seed, vegetative portion), as well as the mass or size of the oil-producing tissue per plant or per growth period.

As used herein the phrase “plant biomass” refers to the amount (e.g., measured in grams of air-dry tissue) of a tissue produced from the plant in a growing season, which could also determine or affect the plant yield or the yield per growing area. An increase in plant biomass can be in the whole plant or in parts thereof such as aboveground (harvestable) parts, vegetative biomass, roots and seeds.

As used herein the term “root biomass” refers to the total weight of the plant's root(s). Root biomass can be determined directly by weighing the total root material (fresh and/or dry weight) of a plant.

Additional or alternatively, the root biomass can be indirectly determined by measuring root coverage, root density and/or root length of a plant.

It should be noted that plants having a larger root coverage exhibit higher fertilizer (e.g., nitrogen) use efficiency and/or higher water use efficiency as compared to plants with a smaller root coverage.

As used herein the phrase “root coverage” refers to the total area or volume of soil or of any plant-growing medium encompassed by the roots of a plant.

The terms “substitution,” “insertion” or “addition,” and “deletion” are used herein with reference to amino acid or nucleotide sequences. A “substitution” refers to a replacement of one or more nucleotides or amino acids by different nucleotides or amino acids, respectively. An “insertion” or “addition” is that change in a nucleotide or amino acid sequence which has resulted in the addition of one or more nucleotides or amino acid residues, respectively, as compared to the naturally occurring sequence. A “deletion” is defined as a change in either nucleotide or amino acid sequence in which one or more nucleotides or amino acid residues, respectively, are absent. Amino acid substitutions are typically of single residues; insertions usually will be on the order of from about 1 to 20 amino acids, although considerably larger insertions may be tolerated. Deletions range from about 1 to about 20 residues, although in some cases deletions may be much larger. Substitutions, deletions, insertions, or any combination thereof may be used to arrive at a final variant polypeptide. Generally, a few amino acids are changed to minimize the alteration of the molecule. However, larger changes may be tolerated in certain circumstances. In certain embodiments, amino acid substitutions can be the result of replacing one amino acid with another amino acid having similar structural and/or chemical properties, such as the replacement of an isoleucine with a valine, i.e., conservative amino acid replacements. Insertions or deletions may optionally be in the range of 1 to 5 amino acids. In embodiments, substitutions can be made in accordance with known “conservative substitutions.” A “conservative substitution” refers to the substitution of an amino acid in one class by an amino acid in the same class, where a class is defined by common physicochemical amino acid side chain properties and high substitution frequencies in homologous proteins found in nature. In contrast, in certain embodiments, substitutions are non-conservative. A “non-conservative substitution” refers to the substitution of an amino acid in one class with an amino acid from another class.

As used herein the phrase “photosynthetic capacity” (also known as “Amax”) is a measure of the maximum rate at which leaves are able to fix carbon during photosynthesis. It is typically measured as the amount of carbon dioxide that is fixed per square meter per second, for example as μmol m-2 sec-1. Plants are able to increase their photosynthetic capacity by several modes of action, such as by increasing the total leaves area (e.g., by increase of leaves area, increase in the number of leaves, and increase in plant's vigor, e.g., the ability of the plant to grow new leaves along time course) as well as by increasing the ability of the plant to efficiently execute carbon fixation in the leaves. Hence, the increase in total leaves area can be used as a reliable measurement parameter for photosynthetic capacity increment.

As used herein the phrase “plant vigor” refers to the amount (measured by weight) of tissue produced by the plant in a given time. Hence increased vigor could determine or affect the plant yield or the yield per growing time or growing area. In addition, early vigor (seed and/or seedling) results in improved field stand.

Improving early vigor is an important objective of modem rice breeding programs in both temperate and tropical rice cultivars. Long roots are important for proper soil anchorage in water-seeded rice. Where rice is sown directly into flooded fields, and where plants must emerge rapidly through water, longer shoots are associated with vigor. Where drill-seeding is practiced, longer mesocotyls and coleoptiles are important for good seedling emergence. The ability to engineer early vigor into plants would be of great importance in agriculture. For example, poor early vigor has been a limitation to the introduction of maize (Zea mays L.) hybrids based on Corn Belt germplasm in the European Atlantic.

It should be noted that a plant trait such as yield, growth rate, biomass, vigor, oil content, fiber yield, fiber quality, fiber length, photosynthetic capacity, fertilizer use efficiency (e.g., nitrogen use efficiency) can be determined under stress (e.g., abiotic stress, nitrogen-limiting conditions) and/or non-stress (normal) conditions.

As used herein, the phrase “non-stress conditions” refers to the growth conditions (e.g., water, temperature, light-dark cycles, humidity, salt concentration, fertilizer concentration in soil, nutrient supply such as nitrogen, phosphorous and/or potassium), that do not significantly go beyond the everyday climatic and other abiotic conditions that plants may encounter, and which allow optimal growth, metabolism, reproduction and/or viability of a plant at any stage in its life cycle (e.g., in a crop plant from seed to a mature plant and back to seed again). Persons skilled in the art are aware of normal soil conditions and climatic conditions for a given plant in a given geographic location. It should be noted that while the non-stress conditions may include some mild variations from the optimal conditions (which vary from one type/species of a plant to another). such variations do not cause the plant to cease growing without the capacity to resume growth.

The term “fiber” is usually inclusive of thick-walled conducting cells such as vessels and tracheids and to fibrillar aggregates of many individual fiber cells. Hence, the term “fiber” includes lignin and also refers to (a) thick-walled conducting and non-conducting cells of the xylem; (b) fibers of extraxylary origin, including those from phloem, bark, ground tissue, and epidermis; and (c) fibers from stems, leaves, roots, seeds, and flowers or inflorescences (such as those of Sorghum vulgare used in the manufacture of brushes and brooms).

Example of fiber producing plants, include, but are not limited to, agricultural crops such as cotton, silk cotton tree (Kapok, Ceiba pentandra), desert willow, creosote bush, winterfat, balsa, kenaf, roselle, jute, sisal abaca, flax, corn, sugar cane, hemp, ramie, kapok, coir, bamboo, Spanish moss and Agave spp. (e.g. sisal).

As used herein the phrase “fiber quality” refers to at least one fiber parameter which is agriculturally desired, or required in the fiber industry (further described hereinbelow). Examples of such parameters, include but are not limited to, fiber length, fiber strength, fiber fitness, fiber weight per unit length, maturity ratio and uniformity (further described hereinbelow).

Cotton fiber (lint) quality is typically measured according to fiber length, strength and fineness. Accordingly, the lint quality is considered higher when the fiber is longer, stronger and finer.

As used herein the phrase “fiber yield” refers to the amount or quantity of fibers produced from the fiber producing plant.

As mentioned hereinabove, transgenic plants of the present invention can be used for improving myriad of commercially desired traits which are all interrelated as is discussed hereinbelow.

As used herein the term “trait” refers to a characteristic or quality of a plant which may overall (either directly or indirectly) improve the commercial value of the plant.

As used herein the term “increasing” refers to at least about 2%, at least about 3%, at least about 4%, at least about 5%, at least about 10%, at least about 15%, at least about 20%, at least about 30%, at least about 40%, at least about 50%, at least about 60%, at least about 70%, at least about 80%, increase in the trait [e.g., yield, seed yield, biomass, growth rate, vigor, oil content, fiber yield, fiber quality, fiber length, photosynthetic capacity, abiotic stress tolerance, and/or nitrogen use efficiency] of a plant as compared to a native plant or a wild type plant [i.e., a plant not modified with the biomolecules (polynucleotide or poly peptides) of the invention, e.g., a non-transformed plant of the same species which is grown under the same (e. g .. identical) growth conditions].

As used herein, “sequence identity” or “identity” in the context of two nucleic acid or polypeptide sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff J G. [Amino acid substitution matrices from protein blocks. Proc. Natl. Acad. Sci. U.S.A. 1992, 89(22): 10915-9]. Identity (e.g., percent homology) can be determined using any homology comparison software, including for example, the BlastN software of the National Center of Biotechnology Information (NCBI) such as by using default parameters.

### Engineering a Permissive Chromatin State

m6A is the most common RNA modification in mammalian cells and controls, among others, translation, decay and chromatin state. The m6A landscape is shaped by m6A writers and erasers, while the effect of m6A modification is generally determined by readers. FTO is an alpha-ketoglutarate (a-KG)-dependent oxygenase, which functions as a m6A demethylase (eraser), making m6A modifications reversible. In addition to m6A, FTO mediates demethylation of N6,2′-O-demethyladenosine(m6Am) and N1-methyladenosine (m1A). Studies have shown the important role of m6A in regulating embryonic stem cell fate as well as other developmental impacts.

By altering the methylation of DNA and/or RNA a permissive chromatin state is achieved in plants. As a function of typical plant reproduction, that permissive state is passed to successive generations of said plant. The surprising discovery has been made that methylation may be achieved without genetic insertion, and through various forms of editing, and such change persists through a set of generations of plants. This achieves a persistent permissive chromatin state in plants that are agronomically beneficial.

The term “vector” as used herein refers to a DNA or RNA molecule capable of replication in a host cell and/or to which another DNA or RNA segment can be operatively linked so as to bring about replication of the attached segment. A plasmid is an exemplary vector.

### EXAMPLES

### Example 1—Assessing First Generation FTO+Plants

A process for assessing generational phenotypic effects will be tested on B. rapa model plants. B rapa plants will be grown following the Fast Plant Growth Protocol. A subset of grown plants will be transformed to express hFTO and mhFTO. Following transformation, FTO expressing model plants, and non-modified first generation plants were outcrossed and collected. Yield, growth rate. biomass, vigor, oil content, seed yield, fiber yield, fiber quality, fiber length, photosynthetic capacity, and/or abiotic stress tolerance will be assessed. Compared to wild type plants, FTO transformed B. rapa plants will be expected to present preferential growth in one or more of assessed assays.

### Example 2—Assessing Second Generation Plants

A process for assessing generational phenotypic effects will be tested on B. rapa model plants. Following transformation of B rapa plants with a demethylase gene such as FTO, second generation cultivars will be grown, where second generation plants are the result of outcrossing from demethylase transformed plants. Yield, growth rate, biomass, vigor, oil content, seed yield, fiber yield, fiber quality, fiber length, photosynthetic capacity, and/or abiotic stress tolerance will be assessed. Compared to wild type plants, FTO transformed B. rapa plants will be expected to present preferential growth in one or more of assessed assays. Moreover, assessed categories will be expected to be similar to transformed parent phenotypes.

A general step-wise protocol is presented below:
(a) Construction of Transgenic Plants that Express FTO
(b) Expression of FTO in plants or plant cells
(c) Phenotyping plants expressing FTO
(d) Recovery, Selfing, and Outcrossing Progeny of FTO expressing plants
(e) Identifying null segregants
(f) Phenotyping null segregants

1. A method for producing a plant exhibiting one or more of increased yield, increased biomass, drought tolerance, salinity tolerance, increased lignin production, biotic stress resistance, abiotic stress resistance, enhanced growth rate, or beneficial root architecture, the method comprising:
(a) expressing a(n) RNA demethylase gene(s) in a first parental plant cell;
(b) outcrossing the parental plant of step (a) with a progeny of said parental plant of step (a) or a plant obtained from said plant cell of step (a), or progeny of a plant obtained from said plant cell of step (a) to a second plant wherein the RNA demethylase gene is not expressed;
(c) screening a population of progeny plants obtained from said outcross of step (b) for one or more of increased yield, increased biomass, drought tolerance, salinity tolerance, increased lignin production, biotic stress resistance, abiotic stress resistance, enhanced growth rate, or beneficial root architecture, wherein a portion of said population of plants expresses the RNA demethylase gene; and
(d) selecting a progeny plant comprising one or more of increased yield, increased biomass, drought tolerance, salinity tolerance, increased lignin production, biotic stress resistance, abiotic stress resistance, enhanced growth rate, or beneficial root architecture that does not express the RNA demethylase gene, wherein one or more of increased yield, increased biomass, drought tolerance, salinity tolerance, increased lignin production, biotic stress resistance, abiotic stress resistance, enhanced growth rate, or beneficial root architecture is heritable and reversible.

2. The method of claim 1, wherein said method further comprises the step of producing seed from: i) a selected progeny plant of step (d); ii) an out-crossed progeny plant of step (d), or, iii) from both of selected and out-crossed progeny plant of step (d).

3. The method of claim 1, wherein the RNA demethylase gene is an FTO.

4. The method of claim 1, wherein the plant expresses the RNA demethylase through the insertion of an RNA demethylase gene via Agrobacterium, gene gun, callous transformation, CRISPR, TALENs, or zinc fingers.

5. The method of claim 1, wherein the plant expresses the RNA demethylase through the use of base-pair editing.

6. The method of claim 5, wherein base-pair editing is achieved through, DNA, RNA or RNA base editing.

7. The method of claim 6, wherein the RNA or RNA base editing further comprises enzymes that target the RNA demethylase and are expressed transiently.

8. An artificial nucleic acid construct, wherein the artificial nucleic acid construct comprises an RNA demethylase, the artificial nucleic acid independently comprising between 400 and 1100 nucleotides or nucleosides or a combination thereof; and when integrated within a plant, the artificial nucleic acid construct is configured to facilitate an epigenetic modification of at least one base of a ribonucleotide or ribonucleoside in a ribonucleic acid of the plant.

9. The artificial nucleic acid construct of claim 8, wherein the RNA demethylase is an FTO gene.

10. The artificial nucleic acid construct of claim 9, wherein a progeny of said plant does not comprise the artificial nucleic acid construct, but comprises the epigenetic modification of at least one base of a ribonucleotide or ribonucleoside in a ribonucleic acid of said plant.

11. The artificial nucleic acid construct of claim 10, wherein the plant is a crop.

12. The artificial nucleic acid construct of claim 11, wherein the crop is soy, maize, wheat, rice, camelina, canola, sugarcane, cucurbits, strawberries, or setaria.

13. The artificial nucleic acid construct of claim 8, wherein the artificial nucleic acid construct is introduced into the plant by a callous phase transformation.
